(19) United States
(12) Patent Application Publication
(10) Pub. No.: US 2008/0194643 A1
(43) Pub. Date: Aug. 14, 2008

(54) Factor Xa Inhibitor Crystalline Forms

(75) Inventors: Brian Samas, Ann Arbor, MI (US); Derek Vrieze, Ann Arbor, MI (US)

Correspondence Address: PFIZER INC., PATENT DEPARTMENT, MS8260-1611, EASTERN POINT ROAD, GROTON, CT, 06340, US

(73) Assignee: PFZER INC

(21) Appl. No.: 11/909,368

(22) PCT Filed: Mar. 13, 2006
(86) PCT No.: PCT/IB2006/000633

§ 371 Date: Sep. 21, 2007

Related U.S. Application Data
(60) Provisional application No. 60/664,870, filed on Mar. 24, 2005.

(51) Int. Cl. A61K 31/4439; C07D 401/12; A61P 7/02; A61P 9/10; A61P 35/00; A61P 3/10

(52) U.S. Cl. 514/343; 546/278.4

(57) ABSTRACT

Disclosed are crystalline forms A, B and C of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). These crystalline forms are characterized by their powder X-ray diffraction, solid-state NMR, as well as methods for the preparation and pharmaceutical compositions of the same which are useful for the treatment of acute, subacute, or chronic thrombotic disorders including treatment of venous thrombosis, arterial thrombosis, pulmonary embolism, myocardial infarction, cerebral infarction, restenosis, atherosclerosis, angina, primary and secondary deep vein thrombosis, thromboembolism associated with cardiovascular disease, including, but not limited to, acute coronary syndrome, atrial fibrillation, cardiac valve replacement and deep vein thrombosis. The crystalline forms of the present invention are also useful for the treatment of cancer, sepsis and diabetes.

### FIELD OF THE INVENTION

The present invention relates to crystalline forms of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) that display inhibitory effects on the serine protease factor Xa. In particular, the present invention relates to crystalline forms A, B and C of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) and methods of using them as therapeutic agents for treating diseases, characterized by abnormal thrombosis, in mammals.

### BACKGROUND OF THE INVENTION

Ischemic heart disease and cerebrovascular disease are the leading causes of death in the world. Abnormal coagulation and inappropriate thrombus formation within blood vessels precipitate many acute cardiovascular diseases.

Thrombin can be considered the key or principal regulatory enzyme in the coagulation cascade; it serves a pluralistic role as both a positive and negative feedback regulator in normal hemostasis. However, in some pathologic conditions, the positive feedback regulation is amplified through catalytic activation of cofactors required for thrombin generation. Such cofactors include factor Xa, a serine protease that occupies a pivotal position in the coagulation cascade.

Abnormal coagulation and inappropriate thrombus formation within blood vessels precipitates many cardiovascular diseases such as myocardial infarction, myocardial ischemia, stroke in association with atrial fibrillation, deep venous thrombosis (DVT), pulmonary embolism, cerebral ischemia or infarction, peripheral artery disease, restenosis, atherosclerosis and thromboembolism. In addition, thrombosis has been linked with non-cardiovascular diseases such as cancer, diabetes and sepsis. Currently some of these conditions are treated with anti-thrombotic agents. However, many of these agents require close monitoring of the patient to protect against bleeding. Recently, it has been appreciated that factor Xa inhibition may provide sustained antithrombotic protection. In animal studies, short term exposure to factor Xa inhibitors produce a sustained antithrombotic effect. Data indicate that factor Xa inhibition potentially provides a large therapeutic window between antithrombotic efficacy and bleeding tendency. Consequently, there may exist a range in which factor Xa inhibition is achieved without a concurrent increase in a patients' susceptibility to bleeding, unlike currently available drugs.

Sepsis is a complex extension of acute inflammation and involves a cycle of progressive amplification of coagulation and inflammation. The intimate involvement of the coagulation system in the progression of this disease has led to treatments that include antithrombotic agents. However, currently available antithrombotic agents do no provide adequate treatment of the disease.

There is a well-known connection between malignancy and thrombosis. Recent evidence has shown that Factor Xa plays a role in tumor metastasis independent from its role in thrombosis and hemostasis.

Type 2 diabetic patients without previous clinical coronary artery disease have the same probability of dying from coronary disease as non-diabetic subjects who have had a previous myocardial infarction. The increased cardiovascular risk in diabetes is contributed to by the clustering of cardiovascular risk factors, which include hypertension, dyslipidemia, hyperinsulinemia, hyperglycemia, obesity, and haemostatic risk factors such as hyperfibrinogenemia and increased levels of plasminogen activator inhibitor-1. These risk factors combine to yield life-threatening thrombotic conditions that could effectively be reduced by treatment with Factor Xa inhibitors.

Factor Xa inhibitors are known in the art, and one compound, ximelgatran, has recently been approved for sale in Europe. However, it is readily apparent that there still exists a need for more effective agents that regulate factor Xa proteolytic activity.

U.S. Patent Application No. US 2003/0162787 A1 to Bigge et al. (the '787 application) describes a number of methods for preparing cyclic amino acid and proline derivatives that inhibit factor Xa. Example 150 describes more specifically the synthesis of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). (Referred to in the '787 application as (2R,4R) 4-Methoxy-pyrrolidine-1,2-dicarboxylic acid 1-[(4-chloro-phenyl)-amide]2-{[2-fluoro-4-(2-oxo-2H-pyridin-1-yl)-phenyl]-amide.)

The chemical and physical properties are important in commercial development of a pharmaceutical compound. These properties include, but are not limited to: (1) packing properties such as molar volume, density and hygroscopicity, (2) thermodynamic properties such as melting temperature, vapor pressure and solubility, (3) kinetic properties such as dissolution rate and stability (including stability at ambient conditions, especially to moisture and under storage conditions), (4) surface properties such as surface area, wettability, interfacial tension and shape, (5) mechanical properties such as hardness, tensile strength, compactibility, handling, flow and blend, (6) filtration properties and (7) bioavailability. These properties can affect, for example, the processing and storage of compositions comprising 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl).

Crystalline forms of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) that provide an improvement in one or more of these properties relative to the non-crystalline forms of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) are desirable in order to improve upon these chemical and physical properties.

In the course of drug development, it is generally assumed to be important to discover the most stable crystalline form of the drug. This most stable crystalline form is the form that is likely to have the best chemical stability, and thus the longest shelf life in a formulation. However, it is also advantageous to have multiple forms of a drug, e.g. salts, hydrates, crystalline and noncrystalline forms. There is no one ideal physical form of a drug because different physical forms provide different advantages. The search for the most stable form and for such other forms is arduous and the outcome is unpredictable.

We have now surprisingly and unexpectedly found crystalline forms of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) designated Forms A, B and C.

### BRIEF SUMMARY OF THE INVENTION

Accordingly, the present invention encompasses crystalline forms of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl).

The formula of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) is shown below.

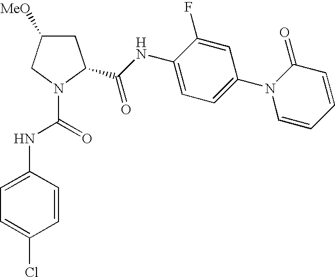

One embodiment of the present invention is the crystalline Form A of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) (Form A). Form A is characterized by the x-ray powder diffraction (PXRD) pattern (Table 1) and/or nuclear magnetic resonance (NMR) spectra (Table 4.)

Another embodiment of the present invention is the crystalline Form B of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) (Form B). Form B is characterized by the x-ray powder diffraction (PXRD) pattern (Table 2) and/or nuclear magnetic resonance (NMR) spectra (Table 4.)

Another embodiment of the present invention is the crystalline Form C of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) (Form C). Form C is characterized by the x-ray powder diffraction (PXRD) pattern (Table 3) and/or nuclear magnetic resonance (NMR) spectra (Table 4.)

Other embodiments of the present invention include, but are not limited to: A crystalline form having a powder X-ray diffraction pattern with at least one peak at 6.0, 16.1, 19.7, 23.2 or 25.4 degrees 2θ;

A crystalline form having a powder X-ray diffraction pattern with peaks at 19.7 and 23.2 and one or more additional peaks at 16.1 or 21.9 degrees 2θ;

a crystalline form having a powder X-ray diffraction pattern with peaks at 19.7 and 23.2 degrees 2θ and having one or more solid-state NMR chemical shifts at 173.8 or 111.3 ppm;

a crystalline form having a powder X-ray diffraction pattern with at least one peak at 16.1, 19.7 or 21.9 degrees 2θ and having one or more solid state NMR chemical shifts at 173.8 or 111.3 ppm;

a crystalline form having a powder X-ray diffraction pattern with at least one peak at 18.9, 25.9, 26.0, 28.7 or 34.8 degrees 2θ;

a crystalline form having a powder X-ray diffraction pattern with peaks at 26.0 and 25.9 degrees 2θ and one or more additional peaks at 18.9 or 21.8 degrees 2θ;

a crystalline form having a powder X-ray diffraction pattern with peaks at 25.9 and 26.0 degrees 2θ and having one or more solid state NMR chemical shifts at 172.9 or 110.0 ppm;

a crystalline form having a powder X-ray diffraction pattern with at least one peak at 18.9, or 21.8 degrees 2θ and having one or more solid state NMR chemical shifts at 172.9 or 110.0 ppm;

a crystalline form having a powder X-ray diffraction pattern with at least one peak at 13.5, or 17.6 degrees 2θ;

a crystalline form of having a powder X-ray diffraction pattern with peaks at 13.5 and 17.6, degrees 2θ and one or more additional peaks at 9.2, 18.3 or 22.5 degrees 2θ;

a crystalline form having a powder X-ray diffraction pattern with peaks at 13.5 and 17.6 degrees 2θ and having one or more solid state NMR chemical shifts at 174.3, 105.4 or 130.3 ppm;

a crystalline form having a powder X-ray diffraction pattern with at least one peak at 9.2, 13.5, 17.6, 18.3, or 22.5 degrees 2θ and having one or more solid state NMR chemical shifts at 174.3, 105.4 or 130.3 ppm;

Another embodiment of the invention is a composition comprising one or more of the above described forms along with a pharmaceutically acceptable excipient, diluent or carrier.

Another embodiment of the invention is a composition comprising one or more of the above described forms along with a pharmaceutically acceptable excipient, diluent or carrier and one or more of the following agents: non-steroidal anti-inflammatory agents, thrombin inhibitors, factor VIIa inhibitors, platelet aggregation inhibitors, vitamin K antagonists, GPIIbIIIa antagonists, heparanoids, thrombolytic and fibrinolytic agents.

A more specific embodiment of the invention is the composition described above wherein the non-steroidal anti-inflammatory agent is one of the following: aspirin, ibuprofen, naproxen sodium, indomethacin, celocoxib, valdecoxib or piroxica. The thrombin inhibitor is one of the following: agatroban effegatran, inogatran, hirudin, hirulog, ximelagatranor or melagatran. The platelet aggregation inhibitor is one of the following: dipyrimidole, aggrenox, clopidogrel, ticlopidine, or a P2Y12 inhibitor. The vitamin K antagonist is one of the following: coumadin, warfarin or a coumarin derivative. The GPIIbIIIa antagonist a is one of the following: abciximab, eptifibitide or tirofiban. The heparanoid is heparin, fraxiparin, tinzaparin, idraparanux, dermatan sulfate, fondaparinux or enoxaparin. The thrombolytic or fibrinolytic agent is one of the following: tissue plasminogen activator, urokinase, streptokinase, plasminogen activator inhibitor-1 inhibitor or thrombin activatable fibrinolysis inhibitor inhibitors.

A crystalline form or a mixture of the forms of the invention can be administered to a mammal in a therapeutically effective amount where use of a Factor Xa inhibitor is indicated. Mammal as used herein includes, but is not limited to, human.

Other embodiments of the invention include, but are not limited to: A method for the treatment of acute, subacute, or chronic thrombotic disorders in a mammal with a therapeutically effective amount of a crystalline form or composition of the invention.

A method for the treatment of primary deep vein thrombosis or secondary deep vein thrombosis in a mammal with a therapeutically effective amount of a crystalline form or composition of the invention. A method for the treatment of thromboembolic events in a mammal with atrial fibrillation with a therapeutically effective amount of a crystalline form or composition of the invention.

A method for the treatment of venous thrombosis, arterial thrombosis, pulmonary embolism, myocardial infarction, cerebral infarction, restenosis, atherosclerosis, angina, primary deep vein thrombosis, secondary deep vein thrombosis, cancer, sepsis, diabetes or thromboembolism associated with cardiovascular disease in a mammal with a therapeutically effective amount of a crystalline form or composition of the invention.

Other embodiments of the invention include, but are not limited to: the use of at least one of Form A, Form B or Form C in the manufacture of a medicament; the use of a crystalline form or composition of the invention in the manufacture of a medicament for treating a condition, in a mammal, for which a beneficial therapeutic response can be obtained by the inhibition of Factor Xa; the use of a crystalline form or composition of the invention in the manufacture of a medicament for treatment of acute, subacute, or chronic thrombotic disorders; the use of a crystalline form or composition of the invention in the manufacture of a medicament for treatment of primary deep vein thrombosis or secondary deep vein thrombosis; the use of a crystalline form or composition of the invention in the manufacture of a medicament for treatment of thromboembolic events in a mammal with atrial fibrillation; the use of a crystalline form or composition of the invention in the manufacture of a medicament for the treatment of venous thrombosis, arterial thrombosis, pulmonary embolism, myocardial infarction, cerebral infarction, restenosis, atherosclerosis, angina, primary deep vein thrombosis, secondary deep vein thrombosis, cancer, sepsis, diabetes or thromboembolism associated with cardiovascular disease or the use of a crystalline form of the invention in the manufacture of a medicament for treatment of venous thrombosis, arterial thrombosis, pulmonary embolism, myocardial infarction, cerebral infarction, restenosis, atherosclerosis, angina, primary and secondary deep vein thrombosis, cancer, sepsis, diabetes, thromboembolism associated with cardiovascular disease, including, but not limited to, acute coronary syndrome, atrial fibrillation, cardiac valve replacement and deep vein thrombosis.

The crystalline forms and compositions of the invention, or mixtures thereof, may be administered in a unit dosage form contained in a package or kit. The kit includes the unit dosage form and a container. Typically, the kit includes directions for administration of the unit dosage form according to a therapeutic schedule. The directions may include directions advising how to use the kit for the treatment of acute, subacute, and chronic thrombotic disorder including but not limited to: treatment of venous thrombosis, arterial thrombosis, pulmonary embolism, myocardial infarction, cerebral infarction, restenosis, atherosclerosis, angina, primary and secondary deep vein thrombosis, thromboembolism associated with cardiovascular disease, including, but not limited to, acute coronary syndrome, atrial fibrillation, cardiac valve replacement and deep vein thrombosis or for the treatment of cancer, sepsis and diabetes. The container can be in any conventional shape or form as known in the art, for example, a paper box, a glass or plastic bottle, or a blister pack with individual dosage forms pressing out of the back.

### BRIEF DESCRIPTION OF THE DRAWINGS

FIG. 1A Diffractogram of Form A 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl).

FIG. 1B Diffractogram of Form B 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl).

FIG. 1C Diffractogram of Form C 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl).

FIG. 2A Solid-state ¹³C nuclear magnetic resonance spectrum of Form A.

FIG. 2B Solid-state ¹³C nuclear magnetic resonance spectrum of Form B.

FIG. 2C Solid-state ¹³C nuclear magnetic resonance spectrum of Form C.

FIG. 3 Differential scanning calorimetry (DSC) thermogram of Forms A, B and C.

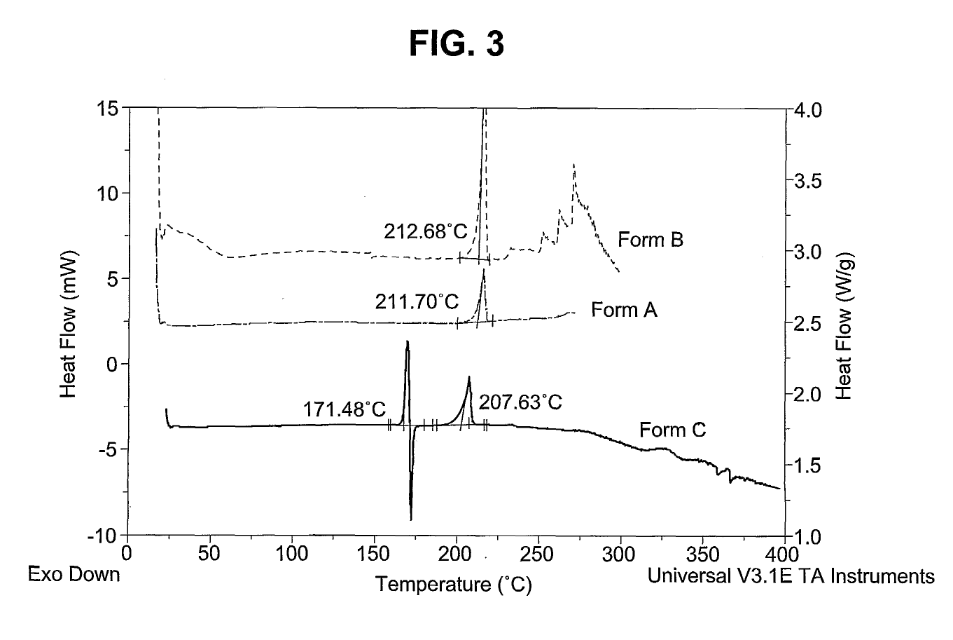

### DETAILED DESCRIPTION OF THE INVENTION

### Definitions

1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) is also known as (2R,4R) 4-Methoxy-pyrrolidine-1,2-dicarboxylic acid 1-[(4-chloro-phenyl)-amide]2-{[2-fluoro-4-(2-oxo-2H-pyridin-1-yl)-phenyl]-amide depending on the nomenclature used to identify the compound. The aforementioned chemical names are used interchangeably and represent the compound shown below.

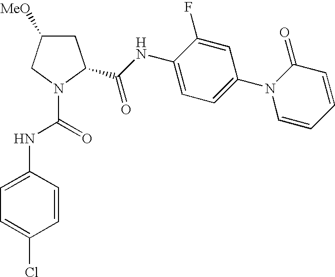

The terms “Form A, Form B and Form C”, as used herein, refer to crystalline forms of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). “Form A”, “Form A polymorph”, “crystalline form A” and “Form A polymorph of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl)” mean the same and are used interchangeably herein. “Form B”, “Form B polymorph”, “crystalline form B” and “Form B polymorph of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl)” mean the same and are used interchangeably herein. “Form C”, “Form C polymorph, “crystalline form C” and “Form C polymorph of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl)” mean the same and are used interchangeably herein.

The term “polymorph” and “crystalline polymorph” and “crystalline form” are used interchangeably herein.

The term “polymorphic form” and “polymorph” are used interchangeably herein.

The term “amorphous” as applied to 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) refers to a solid state wherein the 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) molecules are present in a disordered arrangement and do not form a distinguishable crystal lattice or unit cell.

The term “crystalline form,” “polymorphic form” or “polymorph” as applied to (1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl), refers to a solid state form wherein the molecules of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl), are arranged to form a distinguishable crystal lattice yielding characteristic diffraction peaks when subjected to X-ray radiation.

The term “DSC” means differential scanning calorimetry.

The term “mammal” as used herein includes, but is not limited to, human.

The term “pharmaceutically acceptable” means suitable for use in mammals.

The term “PXRD” means powder X-ray diffraction.

The term “slurry” means a stirred suspension of a solid compound in a solvent wherein that compound is at a higher concentration than its solubility in the solvent. “Slurrying” refers to the making of a slurry.

When used in conjunction with PXRD, the term “pattern” and “diffractogram” as used herein, have the same meaning.

As used herein, the terms “treat”, “treating” and “treatment” and the like, include palliative, curative and prophylactic treatment.

### Powder X-ray Diffraction (PXRD)

Compounds having identical chemical structures may exist in different physical forms. They may be amorphous or may exist as distinct crystalline forms. Different crystalline forms often have different physical properties (i.e. bioavailability, solubility, melting points, etc). These different crystalline forms are sometimes referred to as polymorphs. One method of determining the structure of a crystalline form is referred to as powder X-ray diffraction (PXRD) analysis. PXRD analysis involves collection of crystallographic data from a group of crystals. To perform PXRD analysis, a powdered sample of the crystalline material is placed in a holder that is then placed into a diffractometer. An X-ray beam is directed at the sample, initially at a small angle relative to the plane of the holder, and then moved through an arc that continuously increases the angle between the incident beam and the plane of the holder. The intensity of the reflected radiation is recorded. These data can be expressed in graphical form as a PXRD pattern.

Measurement differences associated with such X-ray powder analyses result from a variety of factors including: (a) errors in sample preparation (e.g. sample height), (b) instrument errors (e.g. flat sample errors), (c) calibration errors, (d) operator errors (including those errors present when determining the peak locations), (e) the nature of the material (e.g. preferred orientation and transparency errors), (f) compound lot to lot differences and (g) machine type. Calibration errors, sample height errors, lot-to-lot variations, and machine type differences often result in a shift of all the peaks in the same direction. These shifts can be identified from the X-ray diffractogram and can be eliminated by compensating for the shift (applying a systematic correction factor to all peak position values) or recalibrating the instrument. This correction factor is, in general, in the range of 0 to 0.2 degrees 2θ.

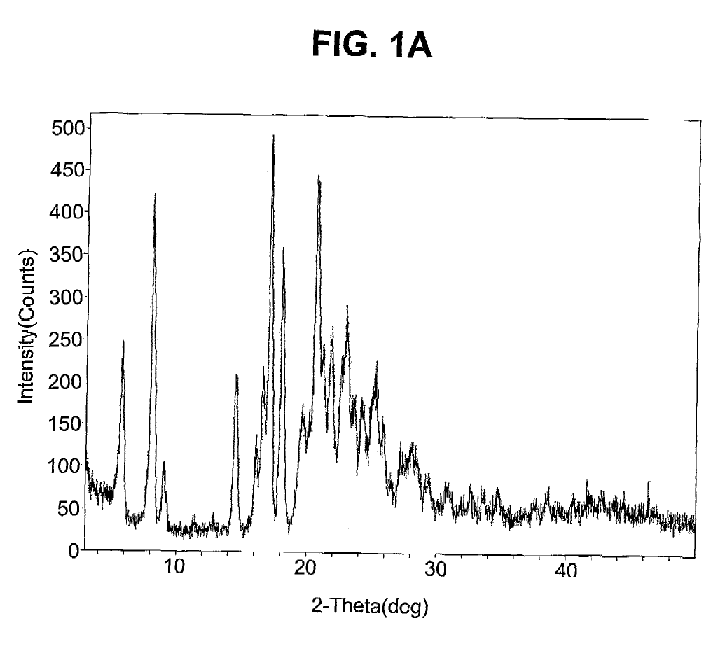

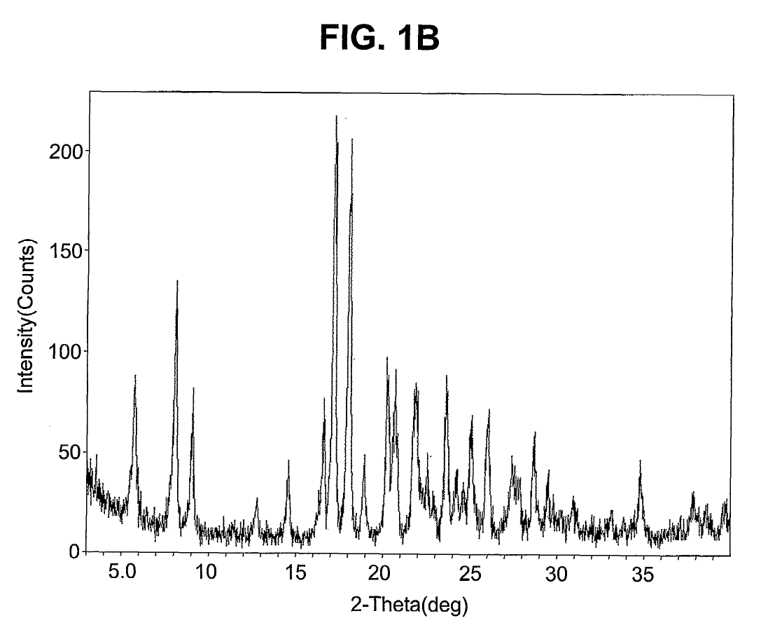

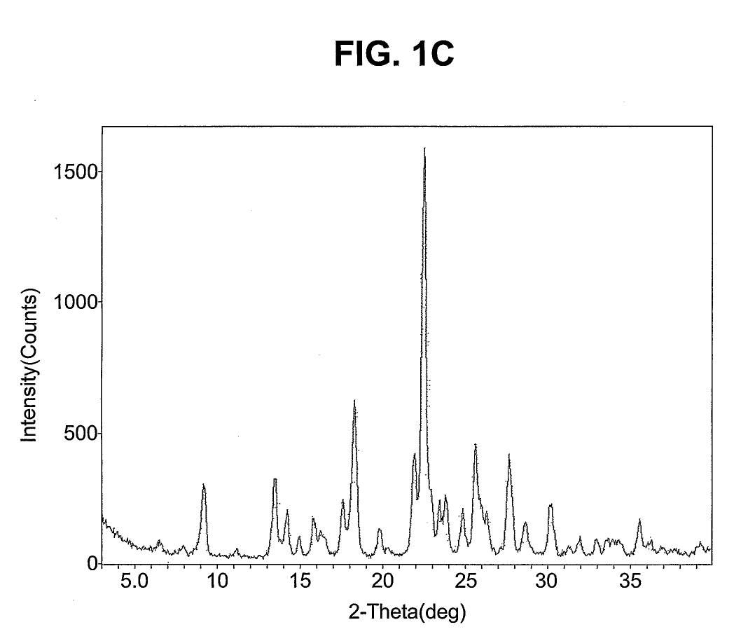

Form A, Form B and Form C 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) are characterized by their PXRD pattern. Samples were prepared for analysis by placing them in an aluminum holder. The powder X-ray diffraction (PXRD) patterns depicted in FIG. 1A, 1B, and 1C were collected on a Rigaku (Tokyo, Japan) Ultima-plus diffractometer with CuKα radiation operating at 40 kV and 40 mA. A NaI scintillation detector detected diffraction radiation. Samples were scanned from 3 degrees to 50 degrees 2-theta at a step size of 0.04 or 0.02 degrees 2-theta at 2.4 seconds per step. An alumina standard was analyzed to check the instrument alignment. Samples were collected at room temperature. Data were processed using Materials Data Inc. Jade (Version 3.1).

Form A is characterized by the PXRD pattern expressed in terms of degree 2θ values and relative intensities with a relative intensity of ≧20.0 (Table 1). Form B is characterized by the PXRD pattern expressed in terms of degree 2θ values and relative intensities with a relative intensity of ≧19.5 (Table 2). Form C is characterized by the PXRD pattern expressed in terms of degree 2θ values and relative intensities with a relative intensity of ≧10.0 (Table 3).

TABLE 1
PXRD Peak List for Form A

| | Relative |
|---|---|
| Degree 2θ | Intensity (%) |
| 5.8 | 43.2 |
| 6.0 | 25.7 |
| 8.1 | 90 |
| 14.6 | 43.5 |
| 16.1 | 22 |
| 16.6 | 39.3 |
| 17.2 | 100.0 |
| 17.3 | 60.3 |
| 18.0 | 73.1 |
| 18.2 | 41.6 |
| 19.7 | 33.4 |
| 20.2 | 24.3 |
| 20.6 | 71.7 |
| 20.8 | 57 |
| 21.7 | 26.9 |
| 21.9 | 31.1 |
| 22.9 | 41.1 |
| 23.2 | 29.7 |
| 25.4 | 21.3 |

TABLE 2
PXRD Peak List for Form B

| | Relative |
|---|---|
| Degree 2θ | Intensity (%) |
| 5.7 | 36.5 |
| 8.1 | 61.5 |
| 9.0 | 36.0 |
| 14.5 | 20.5 |
| 16.6 | 35.0 |
| 17.2 | 100.0 |
| 18.1 | 97.5 |
| 18.9 | 19.5 |
| 20.2 | 44.5 |
| 20.3 | 29.5 |
| 20.7 | 41.5 |
| 21.8 | 36.0 |
| 23.6 | 37.0 |
| 25.0 | 23.5 |
| 25.9 | 25.0 |
| 26.0 | 30.0 |
| 28.7 | 24.0 |
| 34.8 | 20.0 |

TABLE 3
PXRD Peak List for Form C

| | Relative |
|---|---|
| Degree 2θ | Intensity (%) |
| 9.2 | 19.0 |
| 13.5 | 20.7 |
| 14.2 | 11.3 |
| 17.6 | 14.2 |
| 18.3 | 41.0 |
| 22.0 | 26.9 |
| 22.5 | 100.0 |
| 22.8 | 10.4 |
| 23.5 | 13.2 |
| 23.8 | 14.4 |
| 25.6 | 25.8 |
| 26.3 | 10.8 |
| 27.7 | 25.5 |
| 30.2 | 13.0 |

### Solid State Nuclear Magnetic Resonance (NMR)

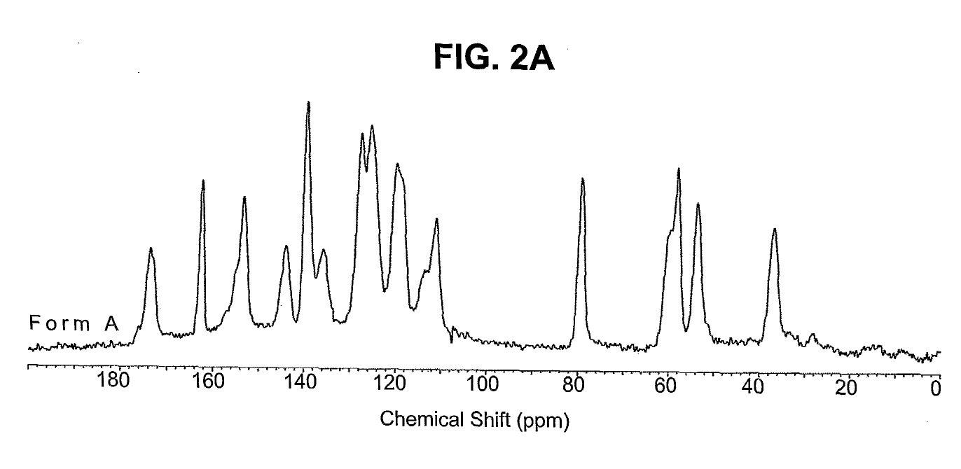

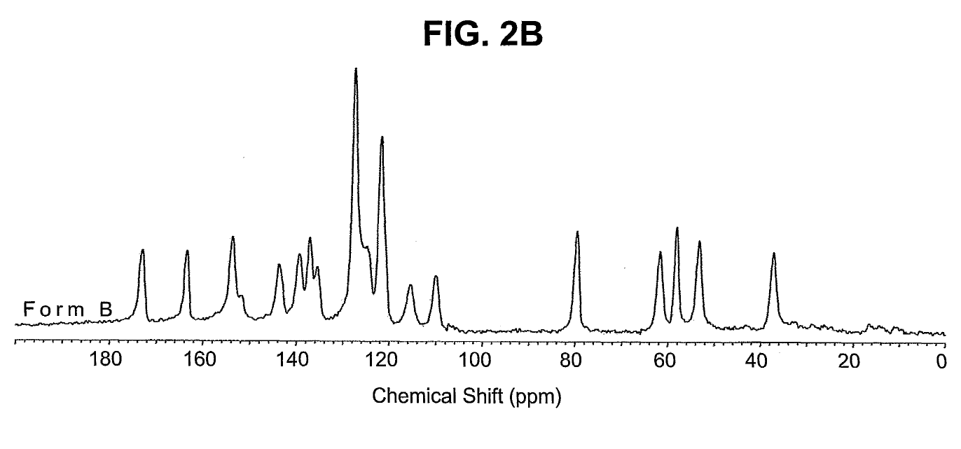

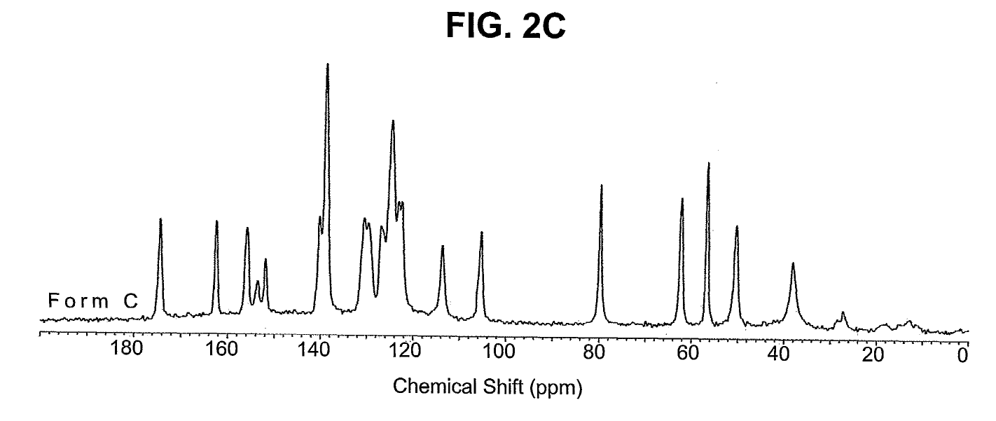

Another method of determining the structure of a crystalline form of a compound is through the use of solid-state NMR. Representative solid-state NMR spectra of Forms A, B and C are shown below in FIGS. 2A, 2B and 2C. Standard acquisition and processing parameters were used. For solid-state NMR, the ¹³C CP/MAS data was acquired at a frequency of 125.65 MHz on a 500 MHz Varian INOVA spectrometer, and externally referenced to the methyl resonance of hexamethylbenzene (17.3 ppm). The spectrometer was equipped with a 2.5 mm Chemagnetics Pencil probe. 3712 data points were acquired over a 46 kHz sweep width. 2048-4096 total transients were acquired. Data was acquired using variable amplitude cross-polarization with a ¹H decoupling field of 140 kHz. Samples were spun at 14 kHz. Tentative chemical shift assignments are listed in Table 4. These assignments were made based on data acquired using an interrupted decouplïng solid-state NMR pulse sequence and information from solution NMR chemical shifts. One skilled in the art would recognize that the chemical shift positions could vary from lot to lot of compound. Also the chemical shift position can vary depending on the instrument used for the measurement. Table 4 gives characteristic shifts for Form A, Form B and Form C.

TABLE 4

| Chem. Shift (Form A) | Chem. Shift (Form B) | Chem. Shift (Form C) |
|---|---|---|
| 173.8 | 172.9 | 174.3 |
| 162.6 | 163.4 | 162.4 |
| 153.5 | 153.7 | 155.8 |
| 144.1 | 143.6 | 153.2 |
| 139.7 | 139.3 | 138.8 |
| 135.9 | 137.1 | 130.3 |
| 127.8 | 135.5 | 124.7 |
| 125.7 | 127.4 | 113.7 |
| 119.9 | 121.7 | 105.4 |
| 111.3 | 115.4 | 79.6 |
| 79.4 | 110.0 | 62.2 |
| 58.2 | 79.5 | 56.4 |
| 53.8 | 61.7 | 50.4 |
| 37.1 | 58.1 | 38.0 |
| | 53.2 | |
| | 37.2 | |

### Differential Scanning Calorimetry (DSC)

Experiments were performed using a DSC 2920 instrument (TA Instruments, New castle, Del.). Nitrogen was used as the purge gas at a flow rate of 50 mL/min for the DSC cell and 110 mL/min for the refrigerated cooling system. The calorimeter was calibrated for temperature and cell constant using indium (melting point 156.61° C., enthalpy of fusion 28.71 J/g). Sealed aluminum pans with a pinhole were used and samples (usually 3-5 mg) were heated at a rate of 10° C./minute. Data analysis was performed using TA Instruments' Universal Analysis 2000 software for Windows Version 3.8B. One skilled in the art would understand that sample purity can alter the characteristics of data obtained by DSC. FIG. 5 shows the DSC traces obtained for forms A, B and C. Depending on experimental conditions the recrystalization and second melt for Form C may not be observed. Temperatures of melting onset for Forms A, B and C are given in Table 5 below.

TABLE 5

| Form | Melting Onset, ° C. |
|---|---|
| A | 211.7 |
| B | 212.7 |
| C | 1^{st} melt: 171.5 |
| | 2^{nd} melt: 207.6 |

The forms of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) described in the present invention, regardless of the extent of water and/or solvent having equivalent PXRD diffractograms are within the scope of the present invention. The present invention provides one or more processes for the preparation of Forms A, B and C 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) which comprises forming a solution or slurry in solvents under conditions which yield Forms A, B or C 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). The precise conditions under which Forms A, B and C are formed may be empirically determined, and it is only possible to give a number of methods which have been found to be suitable in practice.

### Pharmacology, Dose and Formulation

The crystalline forms of the present invention can be administered to a patient at dosage levels in the range of 0.1 to 2,000 mg per day. In another embodiment the crystalline forms of the present invention are administered to a patient in the range of 0.01 to 700 mg per day. In another embodiment the crystalline forms of the present invention are administered to a patient at dosage levels in the range of 0.1 to 300 mg per day. In another embodiment the crystalline forms of the present invention are administered to a patient at dosage levels in the range of 0.1 to 150 mg per day. However, the specific dosage used can vary. For example, the dosage can depend on a numbers of factors including the requirements of the patient, the severity of the condition being treated, and the pharmacological activity of the crystalline form of the compound being used. The determination of optimum dosages for a particular patient is well-known to those skilled in the art.

The crystalline forms of the invention will generally be administered in a mixture with a suitable pharmaceutical excipient, diluent or carrier selected with regard to the intended route of administration and standard pharmaceutical practice. The pharmaceutical preparation may be in a unit dosage form. In such form, the preparation is subdivided into unit doses containing appropriate quantities of the active component. The unit dosage form can be a packaged preparation, the package containing discrete quantities of the preparation, such as packeted tablets, capsules, and powders in vials or ampoules. Also, the unit dosage form can be a capsule, tablet, cachet, or lozenge itself, or it can be the appropriate number of any of these in packaged form.

For example, the crystalline forms of the invention can be administered orally, buccally or sublingually in the form of tablets, capsules, multi-particulates, gels, films, ovules, elixirs, solutions or suspensions, which may contain flavoring or coloring agents, for immediate-, delayed-, modified-, sustained-, pulsed- or controlled-release applications. The crystalline forms of the invention may also be administered as fast-dispersing or fast-dissolving dosage forms or in the form of a high-energy dispersion or as coated particles. Suitable formulations of the crystalline forms of the invention may be in coated or uncoated form, as desired.

Such solid compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

The percentage of the compositions and preparations may, of course, be varied and may conveniently be between 2 to 60% of the weight of a given unit dosage form. The amount of active compound in such therapeutically useful compositions is such that an effective dosage level will be obtained.

The crystalline forms of the present invention are useful for the treatment of acute, subacute, or chronic thrombotic disorders. More specifically the crystalline forms of the present invention are useful for the treatment of venous thrombosis, arterial thrombosis, pulmonary embolism, myocardial infarction, cerebral infarction, restenosis, atherosclerosis, angina, primary and secondary deep vein thrombosis, thromboembolism associated with cardiovascular disease, including, but not limited to, acute coronary syndrome, atrial fibrillation, cardiac valve replacement and deep vein thrombosis. The crystalline forms of the present invention are also useful for the treatment of cancer, sepsis and diabetes.

The crystalline forms are well suited to formulation for convenient administration to mammals for the treatment of such disorders. The crystalline forms of the present invention can be administered alone or in combination with one or more therapeutic agents. These include, for example, other anticoagulants, which include, but are not limited to non-steroidal anti-inflammatory agents including but not limited to, aspirin, ibuprofen, naproxen sodium, indomethacin, celocoxib, valdecoxib and piroxica; thrombin inhibitors including, but not limited to argatroban, effegatran, inogatran, hirudin, hirulog, ximelagatran, and melagatran; vitamin K antagonists including, but not limited to, coumadin, warfarin, and other coumarin derivatives; factor VIIa inhibitors; platelet aggregation inhibitors including but not limited to dipyrimidole, aggrenox, clopidogrel, ticlopidine, or other P2Y12 antagonists; GPIIbIIIa antagonists including but not limited to abciximab, eptifibitide, and tirofiban; heparanoids including but not limited to heparin, fraxiparin, tinzaparin, idraparanux, dermatan sulfate, fondaparinux, enoxaparin; and thrombolytic or fibrinolytic agents such as tissue plasminogen activator, urokinase or streptokinase, plasminogen activator inhibitor-1 inhibitors and thrombin activatable fibrinolysis inhibitor inhibitors. If a combination of active agents is administered, then the agents may be administered simultaneously, separately or sequentially. The following non-limiting examples illustrate methods that may be used in preparing the crystalline forms of the invention.

### EXAMPLES

### Example 1

### (2R,4R)-4-Methoxy-pyrrolidine-2-carboxylic acid [2-fluoro-4-(2-oxo-pyridin-1-yl)-phenyl]-amide

### Step 1. Preparation of (2R,4R)-4-methoxy-pyrrolidine-1,2-dicarboxylic acid 1-tert-butyl ester

A nitrogen-purged, 500 mL, 3-necked flask, which was equipped with a mechanical stirrer and thermocouple, was charged with 60% (w/w) NaH (8 g, 200 mmol) and hexane (250 mL). The mixture was stirred for 1 min, after which the agitation was stopped and the solids were allowed to settle. Hexane was removed with a candle filter. The flask was then charged with THF (250 mL) and CH₃I (6.51 mL, 105 mmol) and the resulting mixture was cooled to 0° C. in an ice bath. (R,R)-4-Hydroxy-pyrrolidine-1,2-dicarboxylic acid 1-tert-butyl ester (22 g, 95 mmol) was then added in portions while maintaining a reaction temperature of 5° C. or less. The reaction was allowed to warm to RT overnight. To the reaction mixture was added H₂O (100 mL), 1N HCl (100 mL) and NaCl (42 g). The reaction was stirred for 10 min. The layers were separated, and the organic layer was dried over MgSO₄, filtered and concentrated to a thick oil. When the solids were just starting to precipitate, hexane (50 mL) was added and a precipitate formed immediately. The mixture was filtered to give the title compound as a white to yellow-white solid (20.16 g). After sitting for a day, the filtrate was filtered to give a second crop of the title compound (1.42 g). The two crops were combined to give the title compound as a white to yellow-white solid (21.58 g, 93% yield; chiral purity via chiral HPLC: 100%).

### Step 2: 1-(4-Amino-3-fluoro-phenyl)-pyridin-2-one

2-Fluoro-4-iodoaniline (10.0 g, 42.2 mmol) was combined with δ-valerolactam (6.27 g, 63.3 mmol), CuI (0.804 g, 4.22 mmol), and K₃PO₄ (22.4 g, 105 mmol). 1,4-Dioxane (60 mL) was added followed by trans-1,2-diaminocyclohexane (1.01 mL, 8.44 mmol). The mixture was heated to reflux for 22 h before cooling and diluting with EtOAc. The mixture was filtered through a plug of silica, eluting with EtOAc, and the filtrate concentrated under reduced pressure. Purification of the crude product by flash chromatography to afford the title compound (3.40 g, 39%) as a brown solid. MS: APCI (AP+): 209.1 (M)+.

### Step 3: (2R,4R)-2-[2-Fluoro-4-(2-oxo-pyridin-1-yl)-phenylcarbamoyl]-4-methoxy-pyrrolidine-1-carboxylic acid tert-butyl ester

Into a solution of the compound of step (1) (0.250 g, 1.02 mmol) in CHCl₃ (10 mL) was added the compound of step (2) (0.212 g, 1.02 mmol), EEDQ (0.302 g, 1.22 mmol), and triethylamine (0.213 mL, 1.53 mmol). The solution was stirred at reflux for 19 h before cooling to RT and adding EtOAc. The solution was washed sequentially with 10% aq. citric acid, 1N NaOH, water, and brine, before drying over MgSO₄ and concentrating under reduced pressure. The crude material was purified by flash chromatography to afford the title compound (0.329 g, 74%) as a tan foam. MS: APCI (AP+): 436.1 (M)+, (AP−): 434.1 (M)−.

### Step 4: (2R,4R)-4-Methoxy-pyrrolidine-2-carboxylic acid [2-fluoro-4-(2-oxo-pyridin-1-yl)-phenyl]-amide

Into a solution of the compound of step 3 (0.329 g, 0.761 mmol) in anhydrous CH₂Cl₂ (5 mL) was added TFA (5 mL). The solution was stirred at RT for 0.5 h before concentrating under reduced pressure to afford the title compound (0.255 g, 100%) as a tan oil.

### Example 2

### Synthesis of Form A from Amorphous 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl)

1.8 g of Amorphous 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) (prepared as described in Example 150 in US 2003/016272787 to Bigge et al.) was slurried in 100 ml of water for 3 days at room temperature. The solids were filtered, washed with 50 ml water and dried in a vacuum oven overnight to give 1.44 g of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). PXRD and DSC confirmed the crystalline form to be Form A.

### Example 3

### Synthesis of Form A from Form B

0.62 g of Form B (Example 4) was stirred in 9.3 ml of methanol and 3.2 ml of water at 50° C. The solution was cooled to room temperature at 5° C./hour. The solids were filtered to give a white solid. The white solid was shown to be crystal form A by PXRD and DSC.

### Example 4

### Synthesis of Crystal Form B from Amorphous 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl)

30.19 g of Amorphous 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) (prepared as described in Example 150 in US 2003/016272787 to Bigge et al.) was heated to reflux in 275 ml of MeOH. 175 ml of water was heated to 80° C. and slowly added to the MeOH/1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) solution. The solution was then cooled to room temperature at 5° C./hour. The solids were filtered, washed with 50 ml of 1:1 MeOH:water and dried in a vacuum oven overnight to give 26.49 g of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). Solids were determined to be Form B by PXRD and DSC.

### Example 5

### Synthesis of Form C from Form B

Synthesis of Form C from Form B 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). Approximately 10 mg of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) was placed in a vial with 2 mL of ethyl acetate. A stir bar was added and left stirring for 3 weeks at room temperature. The solids were filtered and air-dried. Solids were determined to be crystal form C by PXRD and DSC.

### Example 6

### Synthesis of Form C from a Mixture of Form B and Form C

52 g of Form B was slurried in 400 ml of EtOAc with 0.5 g of Form C overnight. The slurry was filtered to give 33.28 g of solids. The solid was confirmed by melting point and PXRD to be Form C.

### Example 7

### Synthesis of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl) and Subsequent Isolation as Form C

2 g of (2R,4R)-4-Methoxy-pyrrolidine-2-carboxylic acid [2-fluoro-4-(2-oxo-pyridin-1-yl)-phenyl]-amide hydrochloride (as prepared in Example 1) was stirred in 17 ml of EtOAc with 0.92 ml of triethylamine for 90 minutes. The reaction mixture was filtered through a celite pad, which was then rinsed with 14 ml of EtOAc. To the reaction filtrate was added 0.86 g of 4-chlorophenyl isocyanate dissolved in 8 ml of EtOAc followed by 10 mg of Form C suspended in 0.25 ml of CH₃CN. The reaction was stirred for two hours, filtered and the solids were washed twice with 6.5 ml EtOAc. The solids were dried in an oven to give 2.17 g of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl). The solids were determined to be Form C by melting point.

### Example 8

### Synthesis of Form B from a Mixture of Form B and Form C

A 1:1 mixture of Forms B and C were slurried in EtOAc at a concentration greater than 25 mg/ml at a temperature greater than 54° C. overnight. The mixture was filtered to give a solid. The solid was determined to be crystal form B by DSC and PXRD.

### Example 9

### Synthesis of Form B from Form C

1.38 g of Form C was heated to 175° C. without solvent and held for 10 minutes. The solid was cooled to room temperature. The solid was confirmed by melting point and DSC to be crystal form B.

What is claimed is:

1. A crystalline form of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl)

2. A crystalline form having a powder X-ray diffraction pattern substantially as shown in FIG. 1A, 1B or 1C.

3. A crystalline form of claim 1 having a powder X-ray diffraction pattern with peaks at 19.7 and 23.2 and one or more additional peaks at 16.1 or 21.9 degrees 2θ.

4. A crystalline form of claim 1 having a powder X-ray diffraction pattern with at least one peak at 16.1, 19.7, 21.9 or 23.2 degrees 2θ and having one or more solid-state NMR chemical shifts at 173.8 or 111.3 ppm.

5. A crystalline form of claim 1 having a powder X-ray diffraction pattern with at least one peak at about 18.9, about 25.9, about 26.0, about 28.7 or about 34.8 degrees 2θ.

7. A crystalline form of claim 5 having one or more solid state NMR chemical shifts at 172.9 or 110.0 ppm.

8. Form C of 1,2-Pyrrolidinedicarboxamide, N1-(4-chlorophenyl)-N2-[2-fluoro-4-(2-oxo-1(2H)-pyridinyl)phenyl]-4-methoxy-, (2R,4R)-(9Cl).

9. A crystalline form of claim 1 having a powder X-ray diffraction pattern with at least one peak at 13.5, or 17.6 degrees 2θ.

10. A crystalline form of claim 9 having a powder X-ray diffraction pattern with peaks at 13.5 and 17.6 degrees 2θ and one or more additional peaks at 9.2, 18.3 or 22.5 degrees 2θ.

11. A crystalline form of claim 9 having a powder X-ray diffraction pattern with peaks at 13.5 and 17.6 degrees 2θ and having one or more solid state NMR chemical shifts at 174.3, 105.4 or 130.3 ppm.

12. A crystalline form of claim 9 having a powder X-ray diffraction pattern with at least one peak at 9.2, 13.5, 17.6, 18.3, or 22.5 degrees 2θ and having one or more solid state NMR chemical shifts at 174.3, 105.4 or 130.3 ppm.

13. A composition comprising a pharmaceutically acceptable excipient, diluent or carrier together with a therapeutically effective amount of a crystalline form of claim 2 or a mixture thereof.

14. A composition according to claim 13 further comprising one or more of the following agents:
a) non-steroidal anti-inflammatory agents;
b) thrombin inhibitors;
c) factor VIIa inhibitors;
d) platelet aggregation inhibitors;
e) vitamin K antagonists;
f) GPIIbIIIa antagonists;
g) heparanoids; and
h) thrombolytic or fibrinolytic agents.

15. A method for the treatment of venous thrombosis, arterial thrombosis, pulmonary embolism, myocardial infarction, cerebral infarction, restenosis, atherosclerosis, angina, primary deep vein thrombosis, secondary deep vein thrombosis, cancer, sepsis, diabetes or thromboembolism associated with cardiovascular disease in a mammal with a therapeutically effective amount of a crystalline form of claim 2 or a mixture thereof.
